(19) United States
(12) Patent Application Publication
(10) Pub. No.: US 2008/0243048 A1
(43) Pub. Date: Oct. 2, 2008

(54) ULTRASOUND WOUND CARE METHOD

(76) Inventor: Eilaz P. Babaev, Minnetonka, MN (US)

Correspondence Address: Bacoustics, LLC, 5929 BAKER ROAD, SUITE 470, MINNETONKA, MN, 55345, US

(21) Appl. No.: 11/692,001

(22) Filed: Mar. 27, 2007

(51) Int. Cl. A61M 37/00

(52) U.S. Cl. 604/22; 601/2

(57) ABSTRACT

The present invention relates to a method of treating wounds by applying ultrasonic energy to a wound to inactivate, destroy, and/or remove infectious agents, and/or deliver a coupling medium to debride, cleanse, and/or sterilize the wound. The method of the present invention comprises the steps of placing the wound in front of the ultrasound emitting surface in near field, activating the ultrasound transducer, generating ultrasonic energy, emitting ultrasonic waves from ultrasound emitting surface, allowing ozone to be created in the near field, applying ozone to the wound, introducing a coupling medium to the ultrasound emitting surface of the wound device, sonicating the coupling medium, and applying the coupling medium to a wound.

### BACKGROUND OF THE INVENTION

The present invention relates to a method of treating wounds, more particularly, a method utilizing ultrasonic energy for effective cleansing, sterilizing debriding, inactivating, destroying, and/or removing infectious agents and/or other contaminants that may be present in a wound.

Wounds encountered in clinical practice can be hard to treat, slow to heal, and difficult to manage. The pain produced by such wounds disables the patient. An unhealed wound's susceptibility to infection increases a patient's morbidity and mortality. These wounds cause patients to experience severe emotional and physical distress as well as creating a significant financial burden on the patients and the healthcare system.

A wound cannot be properly diagnosed until all foreign materials, necrotic tissues, and/or infected tissues are removed. Damaged tissue, necrotic tissue and/or infected tissue must be removed in order to improve the healing potential of the remaining healthy tissue. This removal process is known as debridement. In general, debridement can either be done surgically, mechanically, chemically, and/or with maggot therapy, and these procedures are well-known in the art. These procedures can be tedious and can lead to the accidental removal of healthy tissue. Additionally, these procedures, especially surgical debridement, can lead to further possible complications such as, but not limited to, bleeding, infection and/or delayed healing. The patient experiences great discomfort and pain from the procedures for treating the wound. These procedures fail to sufficiently clean the wound, disinfect the wound, inactivate and/or remove bacteria cells and/or foreign organisms that may be present in the wound.

The method of using ultrasound energy for treating wounds is well-known in the art. Ultrasonic energy is applied to a wound surface by direct contact or indirectly trough a coupling medium. Applying ultrasonic energy to a wound has been known to clean the wound, increase blood flow to the wound, stimulate cell growth, provide medications to the wound and/or penetrate the medications through the surface of the wound. Examples of such methods are utilized in devices disclosed in U.S. Pat. No. 6,478,754 to Babaev; U.S. Pat. No. 6,533,803 to Babaev, U.S. Pat. No. 6,569,099 to Babaev; U.S. Pat. No. 6,663,554 to Babaev; U.S. Pat. No. 6,761,729 to Babaev; U.S. Pat. No. 6,916,296 to Soring et at.; U.S. Pat. No. 6,960,173 to Babaev; U.S. Pat. No. 6,964,647 to Babaev, U.S. Pat. No. 7,025,735 to Soring et al,; and WIPO Patent WO 1997/017933 to Babaev.

In general, wound care methods require ultrasonic energy to be emitted from a radiation surface to a wound surface. A coupling medium may carry ultrasonic energy to a wound from the radiation surface when applied to the wound. Current ultrasonic methods for wound treatments fail to aggressively debride a wound surface, efficiently remove infectious agents, and/or properly and adequately clean the wound.

Since debridement of a wound is crucial to the healing potential of the wound, there is a need for a wound care method that effectively and adequately removes necrotic tissues, damaged tissues and/or infected tissues, supplies drugs to the wound, and/or inactivates, and/or destroys infectious agents that may be present in the wound.

### BRIEF SUMMARY OF THE INVENTION

The present invention is directed towards a method for the treatment of wounds. The method applies ultrasound energy to a wound surface to inactivate and/or destroy infectious agents that may be present in a wound, and/or delivers a coupling medium such as saline, liquids, gels, and/or medications to debride, cleanse, and/or sterilize the wound. The method of the present invention comprises the steps of placing a wound in front of an ultrasound emitting surface, activating the ultrasound transducer, generating ultrasonic energy, emitting ultrasonic waves from an ultrasound emitting surface, allowing ozone to be created in the near field, applying ozone to the wound, introducing a coupling medium to the ultrasound emitting surface of the wound device, sonicating the coupling medium, and/or applying the coupling medium to the wound.

The wound may be placed in front of and in close proximity to the ultrasound emitting surface. Activating the ultrasound transducer generates ultrasound energy that is emitted from the ultrasound emitting surface as ultrasonic waves. Ultrasonic waves emanating from the ultrasound emitting surface may create a near field. Ozone is created in near field and may be delivered to the wound. Ozone may enter the surface of the wound and/or penetrate beneath the wound surface. Ozone may be delivered to a wound through a variety of ways such as, but not limited to, delivery by the ultrasonic waves emanating from a ultrasound emitting surface into the wound, dissolving the ozone in a coupling medium, and/or by diffusion. Ozone inactivates and/or destroys any foreign organisms and/or materials within and beneath the surface of the wound.

Ultrasonic energy may be delivered to the wound using a coupling medium such as, but not limited to, saline, gels, and/or medications. The coupling medium may be used to wash, cleanse and/or sterilize the wound surface. Coupling medium may wash away and/or remove inactivated foreign organisms, materials, and/or bacterial cells. When sonicated liquids are used as the coupling medium, the liquids delivered to the wound irrigates the wound, thereby removing devitalized tissues, necrotic tissues, infected tissues, foreign materials, and/or other contaminants which may impede the healing process of wounds. Ultrasonic energy may also be delivered through the air to the wound and through a coupling medium simultaneously. Delivering ultrasonic energy to the wound increases blood flow to the wound and/or stimulates cell growth.

The method of the present invention is particularly advantageous on a wound surface and surrounding tissues because the delivery of ozone destroys and/or inactivates foreign organisms and/or materials that may be present in the wound. The application of the ultrasound energy to the wound may also increase blood flow, stimulate cell growth, and provide other therapeutic effects to the wound. Applying a sonicated coupling medium to the wound bed washes, cleanses, disinfects and/or sterilizes the wound, delivers medications to the wound, penetrates the medications through the surface of the wound, and/or improves overall healing time of wounds. Another advantage of the present invention is the effective debridement of the wound, which is critical to the healing progress of the wound.

Accordingly, one aspect of the method of the present invention may be to treat wounds and assist in the healing process of wounds.

Another aspect of the method of the present invention may be to inactivate, destroy, and/or remove bacteria cells and/or other infectious agents that may be present in a wound.

Another aspect of the method of the present invention may be to effectively remove necrotic tissues, damaged tissues, infected tissues, and/or other contaminants from a wound.

Another aspect of the method of the present invention may be to deliver coupling medium to a wound.

Another aspect of the method of the present invention may be to sterilize a wound.

Another aspect of the method of the present invention may be to treat narrow wound beds.

Another aspect of the method of the present invention may be to increase blood flow to a wound bed.

These and other aspects of the invention will become more apparent from the written description and figures below.

### BRIEF DESCRIPTION OF THE DRAWING

The present invention will be shown and described with reference to the drawings of preferred embodiments and will be clearly understood in details.

FIG. 1 depicts a flow chart illustrating some possible embodiments of the method of the present invention.

### DETAILED DESCRIPTION OF THE INVENTION

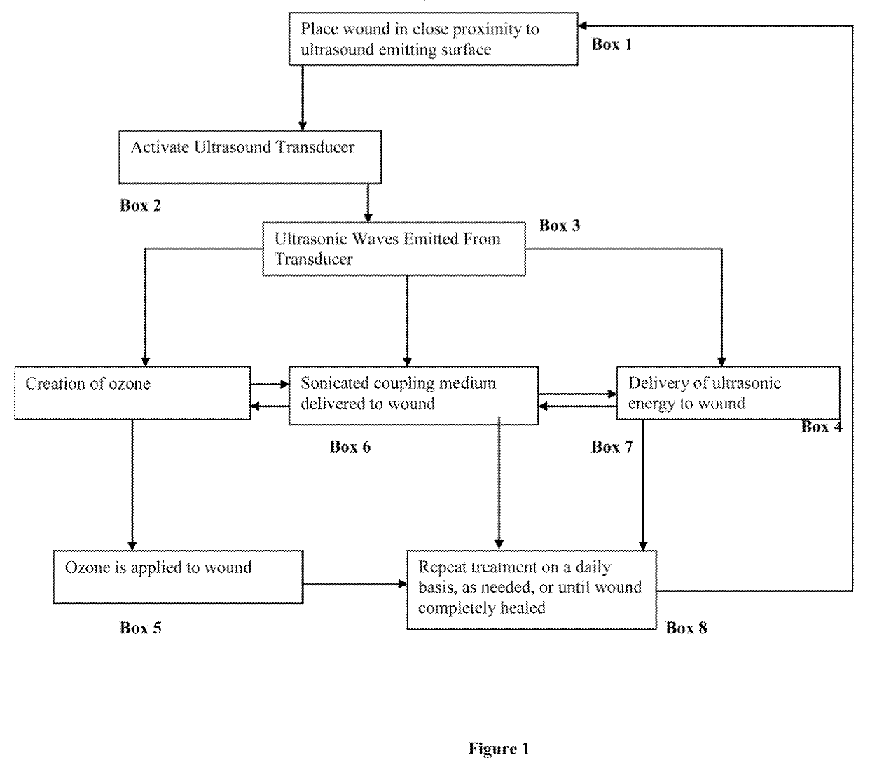

FIG. 1 depicts a flow chart illustrating some possible embodiments of the method of the present invention. The method may begin, as indicated by Box 1, by placing the wound in front of and in close proximity to the ultrasound emitting surface. The reason for placing the radiation surface in front of and in close proximity the wound is to improve the ionization effect of ultrasound waves in near field (Fresnel zone). Ultrasound transducer may be activated, transmitting ultrasound energy through a transducer tip to a radiation surface as indicated by Box 2. It is preferable that the transducer tip is able to move toward the wound and back (x-x) and in direction of the axis of wound (y-y). Ultrasonic waves emnanating from the ultrasound emitting surface, as indicated in Box 3, may create the near field.

According to the theory of classical physics, free electrons are electrons not held in molecular orbit. Negative ions are free electrons. Positive ions are molecules that have lost electrons and are polarized. It is important to notice that significant ultrasonic air ionization process occurs more durable and active in-between the ultrasound emitting surface and a barrier in front of it, such as a wound surface in this present invention. In this condition, ionization of air occurs in the near field-far field interface between the ultrasound emitting surface and the barrier during sonication period.

The length, L, of the near field (Fresnel Zone) is equal to L=r²/λ=d²/4λ, where r is the radius and d is the diameter of the ultrasound emitting surface or distal end diameter of ultrasonic tip, and λ is the ultrasound wavelength in the medium of propagation. Maximum ultrasound intensity occurs at the interface between the near field (Fresnel zone) and the far field (Fraunhofer zone). Beam divergence in the far field results in a continuous loss of ultrasound intensity with distance from the transducer. As the transducer frequency is increased, the wavelength λ decreases, thus resulting in an increase in the length of the near field. Ionization time can be from fractions of a second up to minutes depending on ultrasound energy parameters and design of the ultrasound transducer or tip.

To clarify and describe the ultrasound air ionization effect, stable air (mainly nitrogen and oxygen) molecules are not polarized, and an ultrasound field does not affect them. Air also contains many free electrons (negative ions) that move back and forth in the ultrasound field. Overstressing of air (preferably between ultrasound emitting surface and barrier) at greater than about 1 w/cm² [watts per square centimeter) can cause the free electrons from stable molecules in the air to attain sufficient energy to knock the free electrons from stable molecules in the air. These newly freed electrons knock off even more electrons, producing more negative and positive ions. When the oxygen molecules in the air lose electrons they become polarized positive ions. These positive ions form ozone, as indicated by Box 4:

O₂→O+O

O+O₂>O₃

The fast-moving negative ions, as well as the slower heavy positive ions, bombard the wound surface when applied to the wound, as indicated by Box 5, eventually destroying and/or inactivating foreign organisms, bacterial cells and/or other contaminants that may be present in a wound.

In keeping with FIG. 1, ultrasonic energy emanating as ultrasonic waves may travel through the air to the wound surface, as indicated by Box 7, and may be applied to the wound for a pre-specified period of time such as from fraction of a second to as much as a minute or more, depending on the factors and/or circumstances surrounding the wound. It is preferable that the ultrasound emitting surface does not come in contact with the wound. Ultrasonic waves emanating from the ultrasound emitting surface may be delivered through the air to the wound and may penetrate beneath the surface of the wound. Ultrasonic energy may increase blood flow to the wound and stimulate cell growth in the wound.

A coupling medium such as, but not limited to saline, gels, and/or liquid medications may then be delivered to the wound, as indicated by Box 6. The coupling medium is sonicated as it comes in contact with the ultrasonic waves emanating from the ultrasound emitting surface of a wound care device. Sonicated coupling medium may be delivered to the wound from the ultrasound emitting surface. The sonicated coupling medium may penetrate the wound surface and may be delivered beneath the wound. Sonicated coupling medium flushes the wound surface and/or penetrates beneath the wound, thereby removing dead tissues, damaged tissues, and/or infected tissues and/or any infectious agents that may be present in the wound. Sonicated coupling medium may also deliver drugs and/or medications to the wound. Sonicated coupling medium may also debride the wound, wash, cleanse, and/or sterilize the wound. The above method may be continued on a daily basis, as needed, and/or until wound is completely healed, as indicated by Box 8.

The above description is of an exemplary method for a wound treatment in accordance with the present invention. The above method need not be carried out in the sequence of steps indicated by FIG. 1. Ultrasonic energy may be initially applied to the wound. Sonicated coupling medium may then be delivered to the wound for debridement, to cleanse the wound, and/or to sterilize the wound. The wound may then be placed in front of the ultrasound emitting surface in near field. The ozone created in near field may then be applied to the wound destroying and/or inactivating infectious agents that may be present in the wound and or beneath the wound surface. However, it is preferable ozone applied to the wound initially, and sonicated coupling medium is then delivered to the wound to wash away, cleanse, sterilize and/or remove inactivated and/or destroyed infectious agents and/or other contaminants.

Although specific methods of use have been illustrated and described herein, it will be appreciated by those of ordinary skill in the art that any arrangement that is calculated to achieve the same purpose may be substituted for the specific methods shown. It is to be understood that the above description is intended to be illustrative and not restrictive. Combinations of the above methods of use and other methods of use will be apparent to those having skill in the art upon review of the present disclosure. The scope of the present invention should be determined with reference to the appended claims, along with the full scope of equivalents to which such claims are entitled.

1. (canceled)

2. (canceled)

3: A method for the treatment of wounds comprising the steps of:
generating ultrasound energy within a near field around an ultrasound emitting surface;
placing an ultrasound emitting surface in close proximity to a wound surface;
applying the ultrasound energy to the wound surface;
providing a coupling medium to the ultrasound emitting surface,
delivering the coupling medium to the wound surface;
generating ozone within the near field; and
directing the ozone to the wound surface.

4: The method of claim 3 having an interface between the near field and a far field with the wound surface located near the interface.

5: The method of claim 3 wherein the ultrasound energy ionizes air molecules.

6: The method of claim 3 wherein a coupling medium carries medications to the wound surface.

7: The method of claim 3 wherein a coupling medium debrides the wound surface.

8: The method of claim 3 wherein a coupling medium washes the wound surface.

9: method of claim 3 wherein a coupling medium sterilizes the wound surface.

10: The method of claim 3 wherein the step of applying the ultrasound energy includes moving the ultrasound emitting surface relative to the wound surface.

11: The method of claim 3 having a step of removing infectious agents from the wound surface.
